(19) United States
(12) Patent Application Publication
(10) Pub. No.: US 2005/0058618 A1
(43) Pub. Date: Mar. 17, 2005

(54) Skin, scalp and hair treatment compositions and methods

(76) Inventors: Roy M. Evans, Destin, FL (US); Edward R. Moore, Panama City Beach, FL (US)

Correspondence Address: SYNNESTVEDT & LECHNER, LLP, 2600 ARAMARK TOWER, 1101 MARKET STREET, PHILADELPHIA, PA, 191072950, US

(21) Appl. No.: 10/909,762

(22) Filed: Aug. 2, 2004

Related U.S. Application Data
(60) Provisional application No. 60/491,375, filed on Jul. 31, 2003.

(51) Int. Cl. A61K 7/06; A61K 7/11

(52) U.S. Cl. 424/70.14

(57) ABSTRACT

Disclosed are hair treatment compositions and methods based on the use of, in addition to active treatment compounds, a maintaining agent comprising a reducing saccharide and non-reducing saccharide. In certain preferred embodiments, the weight ratio of non-reducing saccharide to reducing saccharide is from about 9:1 to about 1:2.

### CROSS-REFERENCED TO RELATED APPLICATION

The present application is related to and claims the priority benefit of U.S. provisional application 60/491,375, filed Jul. 31, 2003, which is incorporated herein by reference in its entirety.

### BACKGROUND OF THE INVENTION

1. Field of the Invention

The present invention relates to methods and compositions for the treatment of skin and hair, and more particularly to methods and compositions for cleaning and conditioning the skin, scalp and hair and to methods for altering the shape and/or color of hair.

2. Description of Related Art

Skin and Hair Conditioning and Cleaning Art

Compositions intended for cleaning and/or conditioning skin or hair, particularly of humans, are usually designed to remove accumulated soil and oils from the skin or hair. Unfortunately, the compositions he heretofore used for this purpose have often suffered from a number of disadvantages. For example, one drawback is that many prior compositions have the effect of causing the removal of a large percentage of the oils contained in the skin or hair, which in turn tends to make the skin, the scalp or the hair feel excessively and uncomfortably dry. Another problem with prior cleaning compositions is that one or more components of the composition are irritating to the eyes or even the skin, which is especially troublesome when the products are used on infants, children and adults with sensitive eyes and skin.

Prior solutions to the dryness problem have included the addition of moisturizer to the cleaning or conditioning product. Unfortunately, these solutions often provide a product, which leaves the body with an undesirably oily and slippery residue when the composition is washed from the body. The presence of moisturizers in such compositions sometimes also has a tendency to reduce the dispersibility and foamability of the composition, which can negatively impact the consumer acceptance of the product.

Certain prior compositions have relied on polymers or cationic surfactants to soften hair or skin. It has been observed, however, that following repeated applications of such compositions, the hair and skin may become dull and sticky. U.S. Pat. No. 4,668,508—Grollier, et al. has a recognized this problem and proposed a solution which includes the use of an aqueous dispersion containing a combination of sugar or alkylsugar fatty acid esters and certain reticulated acrylamide copolymers. While such a dispersion is said to result in light and silky hair with considerably improved wet comb through and dry styling properties, applicants believe that even further unexpected and surprising improvement can be achieved in accordance with the methods and compositions of the present invention.

The compositions disclosed in U.S. Pat. No. 4,440,439—Giles, et al. are said to provide improved softening and conditioning effects when incorporated into a hair treatment composition. The disclosed compositions comprise a fatty acid polyester of a polyol selected from cyclic polyols, sugar derivatives and mixtures thereof. While the Giles patent describes monosaccharides and disaccharides as being available for use, and specifically mentions xylose, arabinose, galactose, fructose, sorbose, glucose, maltose, lactose, cellobiose and sucrose, it does not teach a preference for any particular combination of saccharides or that any particular combination has advantages over another.

Hair Coloring Art

Hair treatment compositions are frequently used to alter the color of hair. The capability of precisely producing the desired degree of color change is of primary importance for all hair-coloring techniques. Also of primary importance is the ability of the hair, once altered, to maintain its altered condition for longest possible period of time, thereby reducing the cost and time required to perform additional treatments. However, the ability to fully achieve these objectives has heretofore been limited by several factors. For example, hair-coloring treatments must be effectively completed in the shortest possible period of time in order to be acceptable to the person undergoing the treatment; yet, longer treatment times have heretofore typically been required to achieve the frequently desired deep tones of color. Moreover, many of the components of the compositions used to alter hair color are known to produce offensive skin and/or scalp irritation, especially when used in concentrations adapted to effect the maximum change in color. From the viewpoint of the hair care professional, it is also highly desirable that the treatment techniques be as simple as possible; yet, one-step methods for dyeing hair have heretofore been rare and/or relatively ineffective.

In general, the color of human hair has heretofore been altered either by bleaching, dyeing or a combination thereof. Mammalian fibers, including human hair, are composed of three major components: a cuticle, a cortex and a medulla. The medulla is central to the hair shaft and is wrapped by strands of keratin, which forms the cortex. The cuticle consists of overlapping flat scales covering the cortex. Melanin is the principal pigment responsible for the color of human hair. Chemical bleaching alters hair coloration by the removal and/or alteration of the melanin. This is typically accomplished by applying oxygen-releasing compounds to the hair. Perhaps the most widely used of such compounds is hydrogen peroxide, which is commonly applied in the form of an aqueous solution. Such hydrogen peroxide solutions operate by opening the imbrications of the cuticle, penetrating and attacking the keratin structure and gradually lightening the shade of the hair by oxidizing the melanin. The “lightening” of the hair increases as contact times and hydrogen peroxide concentrations increase. According to prior bleaching methods, however, these conditions also tend to produce scalp irritation and undesirable weakening of the hair shaft. Furthermore, modifying the color of human hair with bleaching agents alone does not necessarily impart the desired color or shade of hair. In particular, bleaching of hair with oxidizing agents alone often results in hair having a “washed out” appearance.

Another method for altering the color of hair involves dyeing the hair. In most hair dyeing techniques, synthetic organic agents are applied to the hair to either temporarily or permanently add color thereto. Examples of temporary or semi-permanent dyes include, for example, azo and nitro compounds, and derivatives of naphthalene and anthraquinone. Semi-permanent dyes are direct dyes and generally do not require any bleaching action to color the hair. However, semi-permanent dyes generally only remain on the hair temporarily and are gradually washed out by successive ordinary treatment of the hair. For example, it is common that ordinary conditioners and shampoos will have a tendency to remove or “wash out” such dyes, thereby severely limiting the longevity of the desired hair color.

Permanent dyes, on the other hand, comprise oxidation dyes, also known in the art as peroxide dyes. Most of these dyes comprise synthetic organic compounds, which generally require an amount of hydrogen peroxide, or some other non-contaminating compound that readily liberates oxygen for the development of the color on the hair. The compounds frequently referred to as oxidation dyes are more properly referred to as dye intermediates, because their actual dyeing properties are developed only upon oxidation. While a large number of compounds posses the potential for being used as dye intermediates for in vivo hair coloring, only a few such compounds have been used according to prior techniques. For example, many nitro and alkyl compounds posses desirable dyeing properties but have been unavailable for use because they are known to irritate the skin. See Wall, F. E., “Bleaches, Hair Colorings, and Dye Removers,” Cosmetics: Science and Technology, Vol. 2, 2nd ed., John Wiley & Sons, p. 307 (1972).

Modification of hair color using a dyeing agent alone also frequently produces less than the desired outcome. For example, the prior hair dyeing techniques are known to frequently result in hair having an unnatural “painted” or “brassy” appearance. This undesirable result has previously been overcome by lightening the hair prior to dyeing by exposing the hair to a bleaching operation. However, this complicates and lengthens the coloring procedure.

To avoid this two-step procedure, compositions containing both a bleaching agent and a dyeing agent have been proposed. These compositions generally contain hydrogen peroxide as both the bleach and the developing agent. These bleach-dye combinations suffer from serious drawbacks. In particular, exact proportions of ingredients are usually required such that a precise amount of oxygen is released to ensure that the hair is bleached while the dye is entering it. Furthermore, excessive release of oxygen results in bleaching of the dye itself. See Wall, F. E., “Bleaches, Hair Colorings, and Dye Removers,” Cosmetics: Science and Technology, Vol. 2, 2nd ed., John Wiley & Sons, pp. 279-343 (1972).

The use of compositions containing disaccharides has heretofore been used for the treatment of hair. For example, U.S. Pat. No. 5,560,750, also incorporated herein by reference, discloses hair-coloring compositions, which incorporate non-reducing disaccharides such as sucrose to improve the hair coloring properties of the dye component and to protect keratin fibers in the hair from damage.

While such compositions have achieved a substantial degree of success, the present inventors have come to appreciate that substantial additional improvement is needed in certain respects. For example, applicants have recognized that the length of time for which the alteration is effective can be substantially improved relative to prior compositions, which had included disaccharides.

Hair Shape Alteration Art

The treatment of human hair to alter its shape has long been an objective of the hair care industry. In order for such treatments to be considered successful, especially for treatments in vivo, many of the same varied and contradictory requirements are present as described above with respect to hair coloring. For example, the capability of precisely producing the desired degree of shape alteration is of primary importance for all hair perming and straightening techniques. However, the ability to fully achieve this objective has heretofore frequently been limited by countervailing requirements. For example, treatments must be effectively completed in the shortest possible period of time in order to be acceptable to the person undergoing the treatment; yet, longer treatment times have heretofore sometimes been required to achieve the desired degree of shape alteration. On the other hand, longer treatment times tend to produce hair damage and offensive skin and/or scalp irritation.

It has heretofore been common practice to treat kinky or curly hair with highly alkaline or caustic aqueous solutions in an effort to soften or reduce the natural elasticity or resiliency of the hair. While the use of such solutions has been partially effective, there are many serious disadvantages as well. For example, methods employing such compositions usually require that the hair and scalp be exposed to highly caustic conditions (e.g.: pH of about 12 to 14) for extended periods of time (e.g.: 45 minutes). Such exposure has heretofore frequently caused a permanent deleterious effect on the aesthetic and structural qualities of the hair and has been known to frequently cause severe irritation or burning of the scalp. See, for example, the article by F. Lewis, M. D., in the Journal of the American Medical Association, Jan. 7, 1939. Moreover, such compositions usually have a very unpleasant odor and are uncomfortable to the person whose hair is being treated. It is also difficult or impossible to alleviate such offensive odors by traditional methods, such as including a pleasant fragrancing agent in the solution since the compounds of the fragrance are usually destroyed or rendered ineffective in highly caustic solutions. It also appears that such compositions have heretofore caused changes in the structure of the hair, which preclude further beneficial chemical treatment of the hair, such as coloring. For example, hair treatment compositions containing sodium or potassium hydroxide, especially at high concentrations of active ingredient, can cause rupture of various linkages and bonds in the protein molecules of the hair to an extent that damage to and embrittlement of the hair occur. For this reason, compositions of this type have required the use of a protective cream which is applied to the scalp and surrounding epidermis prior to application of the shape altering composition. See Wall, F. E., “Hair Straighteners,” Cosmetics: Science and Technology, Vol. 2, 2nd ed., John Wiley & Sons, p. 265 (1972).

Methods are also currently available for imparting a so-called “permanent wave” to hair, which is naturally relatively straight. One frequently used method requires application of a highly alkaline solution to the hair and subsequent physical curling of the hair in the presence of heat. For example, see Brown, U.S. Pat. No. 2,115,156. In addition to the disadvantages described above in connection with the use of highly caustic solutions, another problem with such methods is that they require the use of high heat in close proximity to the skin and scalp, creating the potential for severe burning.

Methods also exist for imparting a permanent wave to hair without the application of heat. Such methods generally require the application of a highly alkaline softening composition to the hair, followed by mechanically conforming the hair to the desired shape. An acidic fixing composition is then applied directly to the conformed and softened hair. The fixing composition neutralizes the softening composition and is said to restore the natural elasticity to the hair. An example of such a method is described in Malone, U.S. Pat. No. 2,061,709. Once again, these methods suffer from all the disadvantages associated with the application of a highly caustic composition to the hair. Moreover, it is difficult if not impossible, to apply the exact amount of acidic fixing composition required to completely neutralize the softening composition without leaving the hair in a slightly acid condition, which in turn will result in an undesirable further softening of the hair.

As recently disclosed in U.S. Pat. Nos. 4,947,878, 5,101,841 and 5,415,856, all incorporated herein by reference, it has been found that safe, effective methods and compositions for the chemical treatment of hair and, more particularly, for imparting a permanent wave to hair without the application of substantial heat thereto, or for otherwise altering the configuration of hair while protecting the hair and scalp from the deleterious effects of exposing the hair to highly caustic solutions, as well as stable aqueous solutions containing cysteine or a similar shaping agent for the treatment of hair, are disclosed that employ a hair shaping composition comprising a shaping agent and a non-reducing disaccharide. According to preferred embodiments disclosed therein, the shaping agent comprises a reducing agent for reducing the hair and a non-reducing disaccharide such as sucrose as a protecting agent to protect the hair and scalp form the caustic compositions. According to other preferred embodiments disclosed therein, the shaping agent comprises alkali. Such compositions have been found to provide excellent ability to cause the desired degree of shape alteration while simultaneously protecting the hair and scalp from burning or damage.

Another method, which has heretofore been used, for the treatment of hair includes mixing cysteine in powder or crystalline form to a treating solution just prior to application of the solution to the hair. The treating solution of such heretofore used methods generally comprised sodium hydroxide solutions having a pH of about 12. Of course, such caustic solutions suffer from all the disadvantages described above, such as the scalp burning and hair damage, which may result from the use thereof. Moreover and just as importantly, cysteine is unstable in aqueous solution and is readily oxidized by dissolved oxygen, thereby precluding long-term storage of the heretofore-used solutions. Another disadvantage is that such methods are cumbersome, inconvenient and may result in an improperly formulated treating solution. It also is relatively expensive to separately and anaerobically package an accurately defined amount of cysteine powder, as would be required to prevent oxidation or decomposition.

U.S. Pat. No. 5,639,451, incorporated herein by reference, discloses methods and compositions for the treatment of hair in which the compositions comprise cysteine and/or another active shaping compound, cysteamine, and a non-reducing disaccharide such as sucrose.

### SUMMARY OF THE INVENTION

Accordingly, one object of certain embodiments of the present invention is to provide hair and/or skin treating compositions which have a relatively long lasting effect and/or which tend to increase the longevity of the effect produced by other treatment compositions and methods.

It is an object of certain embodiments of this invention to provide a cosmetic composition that is exceedingly mild to the skin, and/or the hair and/or to the eyes.

It is an object of certain embodiments of this invention to provide a cleansing composition that does not leave the skin or hair with an excessively dry feeling.

It is an object of certain embodiments of this invention to provide cosmetic compositions with the combination of attributes of mildness to the skin and/or hair and/or eyes and effective treatment while leaving the skin and/or hair with a feel that is neither excessively dry nor oily.

It is a further object of certain embodiments of the invention to provide hair treatment compositions, which are relatively non-irritating to the skin.

It is a still further object of certain embodiments of the invention to provide hair treatment compositions, which do not cause unwanted damage to the hair and are readily adaptable for in vivo use.

It is another object of certain embodiments of the invention to provide effective hair coloring processes that provide bleaching and dyeing in a single step.

Applicants have discovered that these and other objects of the present invention are achieved by providing compositions, and especially hair coloring compositions, comprising a reducing saccharide and non-reducing saccharide. Although it is contemplated that the ratio of reducing saccharide to non-reducing saccharide may vary widely within the scope of the present invention, it is preferred that the weight ratio of non-reducing saccharide to reducing saccharide is from about 99:1 to about 1:99, more preferably from about 9:1 to about 1:2, and even more preferably from about 5:1 to about 1:1. According to preferred embodiments, the reducing saccharide comprises, and preferably consists essentially of, glucose and the non-reducing saccharide comprises, and preferably consists essentially of, sucrose.

The compositions of the present invention are preferably utilized as a maintaining agent for hair and/or skin care compositions. All applicants do not necessarily understand the basis of operation of the present compositions, it has been discovered that the combination of reducing and non-reducing saccharides as described herein produces unexpected but highly beneficial maintenance properties to a wide variety of hair and skin treating compositions. Hair and skin treating compositions which may be adaptable for use in accordance with the present invention include, but are not necessarily limited to, shampoos, conditioners, rinses, setting lotions, gels styling agents, hair tonics, hair creams, detangling agents, colorants, bleaches, dyes, shape altering compositions (including straighteners and perms), split-end reducers, face cleansing creams, toilet waters, emulsions, powders, foundations, lipsticks, rouges, eye cosmetics (including eye shadow, eyeliners and mascara), manicure products, and the like. Applicants have found that in many applications the desired result of hair and/or skin treatment compositions can be maintained for a relatively prolonged period of time compared to the effect of such compositions in the absence of the maintaining agent of the president invention. For example, one aspect of the present invention provides shampoo, conditioning compositions, soaps and the like, which comprise a maintaining agent in accordance with the present invention. It such embodiments, the presence of the maintaining agent and composition tends to place the hair, skin or scalp in a condition such that its desired shape, color, shine, luster, softness, cleanliness, etc. exists for a period of time longer and/or at a higher or more desirable level, than that which would occur in the absence of the maintaining agent. Accordingly, in certain preferred embodiments, the present invention provides a hair or skin treatment composition, such as a shampoo or conditioner, comprising a maintaining agent of the present invention in amounts effective to increase, and preferably substantially increase, more preferably increased by at least about 30%, and even more preferably increased by at least about 50%, the length of time that hair treated with the composition retains its desired color, shape, luster, softness, and/or other aspect of its appearance or feel relative to treatment in the absence of the maintaining agent. In preferred embodiments, the maintaining agent is present in the treatment composition in amounts of from about 0.5% to about 15% by weight, and more preferably from about 1% to about 10% by weight, and even more preferably from about 1% to about 5% by weight.

Preferred method aspects of the present invention comprise the steps of providing a composition comprising a reducing saccharide and a non-reducing saccharide has described herein, preferably incorporated into a hair or skin treatment composition, and applying the composition to the hair or skin. Although it is contemplated that such methods will provide advantage when used in connection with all types and the conditions of hair, applicants have found that especially beneficial results are obtained when the application step of the present invention comprises applying the composition to hair which is undergoing or has undergone a shape and/or color alteration. For example, applicants have found that it is possible to greatly increase the length of time that a desired change in hair color is maintained by applying to the hair a composition of the present invention at about the time the color is being changed, preferably by incorporating the maintaining agent of the present invention into such color altering compositions. Likewise, the maintaining agent of the present invention can be used with great advantage as a component of a composition for altering the shape of hair, or any of the other hair treatment compositions mentioned above.

In another aspect, the present composition is applied to hair or skin that has already undergone alteration or treatment, such as a color change or, in the case of hair, shape alteration. Applicants have found that such methods are especially effective in maintaining the desired color or shape of the hair for a longer period of time than would otherwise occur. As those skilled in the art will appreciate, and as mentioned above, it is common that hair treatment methods produce only a temporary change in the desired change in color or hair shape, with the hair eventually reverting to its original color and/or shape. The methods in compositions of the present invention extend the period of time during which the hair continues to exhibit the desired color and/or shape and/or other desired property, thereby greatly reducing the frequency with which the current or shape altering composition needs to be reapplied to maintain the desired change in hair property. Thus, applicants have found that highly desirable maintenance properties are achieved when the present maintaining agent is incorporated into shampoo, conditioner or the like and such a composition is then applied to the altered hair to clean, condition or treat the hair. Applicants expect that like results are possible with the other type of skin and hair treatment compositions mentioned above.

In certain embodiments, the present invention provides a hair treatment composition including the maintaining agent of the present invention, a coloring agent comprising at least one oxidizing agent for bleaching the hair and/or developing the dyeing agent, and a dyeing agent for adding color to the hair. Such compositions have been found capable of producing exceptionally deep and desirable color, even without pre-bleaching. Furthermore, such compositions have surprisingly been found to be effective after only relatively short periods of application while being exceptionally non-irritating to the skin and scalp, and non-damaging to the hair.

Preferred method aspects of the present invention relate to a process for altering the color of hair to a predetermined degree comprising (a) applying to the hair a hair coloring composition, said composition comprising a coloring agent and a maintaining agent of the present invention; (b) allowing said composition to remain in contact with the hair for a time sufficient to achieve the predetermined degree of color alteration; and (c) substantially removing said composition from the hair.

Furthermore, in accordance with certain aspects of the present invention, it has now been found that the properties of the hair treatment compositions disclosed U.S. Pat. Nos. 4,947,878, 5,101,841, 5,415,856, 5,560,750 and 5,639,451 (each of which is incorporated herein by reference) can be improved by the use of a reducing saccharide in place of the non-reducing disaccharides disclosed therein when the compositions comprise highly caustic components. The reducing saccharides in the maintaining agent protect the hair and scalp from the caustic component, improve performance (especially in applications relating to changing the color of hair), and control the unacceptable odor heretofore associated with such hair care compositions, while the non-reducing saccharide tends to protect the skin and scalp, and the synergistic combination of the two imparts the desirable maintaining property described above.

Preferred reducing saccharides for use in the present invention include disaccharides such as maltose or lactose, and monosaccharides such as glucose, with glucose being generally preferred. Preferred non-reducing saccharides comprise sucrose.

### DETAILED DESCRIPTION OF THE INVENTION

I. Chemistry of Hair

In order to more fully understand the compositions and methods of the present invention, it is helpful to understand the basic structure of hair. Hair is a complex organic substance consisting largely of the protein keratin. More specifically, hair is a proteinaceous fiber comprising a bundle of long individual protein molecules, which are intertwined with one another and cross-linked at various intervals. Each individual protein molecule comprises condensed amino acids in which the acid end of one molecule is condensed with the amino end of the next. The amino acids are alike in that they all contain an acid group and an amino group, but they may not be alike in certain other details of the arrangement of their atoms. Hair protein generally contains from about 5% to about 15% by weight of the amino acid cystine, which has the empirical chemical formula C₆H₁₂N₂O₄S₂ and generally conforms to the molecular chemical formula given below: 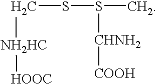
It has been postulated that cystine frequently appears in the fibrous bundle of keratin protein molecules as a bridge between adjacent peptide chains and that it may also frequently appear as a loop or bridge between two segments of the same peptide chain. It is believed that these cystine bridges affect, and in large part determine, the physical shape and conformation of the hair. It has also been postulated that links between adjacent peptide chains may also occur by the ionization of the carboxyl and amino groups to form a salt bridge. Hydrogen bonding is believed to provide a third means by which linkage between adjacent peptide chains may be achieved. It is believed that these additional linkage mechanisms also affect the physical shape and conformation of the hair. See chapter 26 of the book “Chemical and Manufacture of Cosmetics” by Mason G. Denavarre and the first chapter of the book entitled “The Proteins Volume 4”, Third Edition, edited by Hans Nuroff and Robert L. Hill, 1979, both which are incorporated herein by reference.
II. The Coloring Compositions

The present coloring compositions comprise two important ingredients: coloring agent and reducing saccharide. Applicants have found that the inclusion of maintaining agent, and preferably maintaining agent in which the reducing saccharide is a monosaccharide such as glucose, in compositions according to the teachings of the present invention provides the compositions with highly desirable and unexpected properties. In particular, the present compositions have been found to be non-damaging to the hair as compared to prior products. Furthermore, the compositions when used in hair color lightening applications have been found to produce exceptional “lift” to the hair and it to maintain that lift for unexpectedly extended periods of time. More particularly, the present composition have been unexpectedly found to produce a full 35% percent improvement in “lift” and to maintain that lift in certain embodiments for at least about 50% longer than it would be the case in the absence of the maintaining agent. As is known to those skilled in the art, “lift” refers to the degree of lightening that is imparted to hair color as a result of application of the composition.

Furthermore, the present compositions have unexpectedly been found to produce exceptionally deep color to the hair. Although applicants do not wish to be bound by or to any particular theory of operation, it is believed that the synergistic combination of ingredients used in the present invention prevents or at least inhibits autooxidation of the dye molecules contained in the composition, thus tending to color the hair deeply, as opposed to only on the surface of that hair as is apparent with certain prior art products. According to certain embodiments, this desirable color alteration is achieved without the benefit of a pre-bleaching step.

A. The Coloring Agents

The present compositions comprise coloring agent, preferably a major proportion of coloring agent, and more preferably from about 85 to about 99 percent by weight of coloring agent and more preferably from about 95 to about 99 percent by weight of coloring agent. The term coloring agent is used herein in a non-limiting sense to refer to any agent, compound or composition adapted to alter the color of hair. Thus, the coloring agents of the present invention include those compositions, which tend to remove color from the hair as well as those, which add color.

It is contemplated that the coloring agents of the present invention will generally include one or more active color compounds and carrier for the active compounds. It will be appreciated that the terms active color compound and carrier are used herein for the purpose of convenience and illustration but not necessarily by way of limitation. In particular, the term active color compound refers to those components of the coloring agent, which interact chemically or physically with the hair or with other components of the coloring agent to add or remove color from the hair. In contrast, the carrier serves to provide the proper environment for the active compounds and to facilitate, enhance and/or modify delivery and application of the active compounds to the hair. The preferred coloring agents of the present invention comprise from about 3 to about 20 percent by weight of active coloring compounds and from about 80 to about 95 percent by weight of carrier, and even more preferably from about 5 to about 15 percent by weight of active coloring compound and from about 80 to about 90 percent by weight of carrier.

An important aspect of certain embodiments of the present invention resides in the weight ratio of reducing saccharide to active coloring compounds. Applicants believe that such ratio may vary widely, depending upon numerous factors, such as the type of hair and the degree of color alteration desired. It is preferred, however, that the reducing saccharide:active coloring compound weight ratio be from about 0.01:1 to about 1:1, and even more preferably from about 0.02:1 to about 0.05:1. Applicants have found that, according to certain embodiments, ratios of less than about 0.01:1 produce compositions, which exhibit a decreased ability to protect the hair and skin of the user, while compositions having ratios greater than about 1:1 may exhibit a decreased ability to produce the desired color alteration.

1. The Coloring Agents—Active Compounds

According to certain embodiments of the present invention, the coloring agent comprises an oxidizing agent. As will be appreciated by those skilled in the art, oxidizing agents are commonly used as active components for both removing color from and adding color to hair. As the term is used herein, oxidizing agent refers to those compounds, which are oxidizing agents with respect to melanin in the hair or with respect to the dye intermediate used in the oxidative dyeing agents of the present invention. For example, in bleaching operations the oxidizing agent attacks the melanin of the hair and removes color, thereby lightening the hair color. In dyeing operations, an oxidizing agent is typically included to aid in the conversion of the dye intermediates used in permanent dyeing operations. It is seen, therefore, that the amount of oxidizing agent contained in the present compositions will vary widely, depending upon the particular circumstances of each embodiment. It is generally preferred, however, that the present compositions comprise from about 3 to about 20 percent by weight of oxidizing agent. For embodiments in which bleaching and dyeing steps are carried out separately, the compositions preferably comprise from about 10 to about 20 percent by weight of oxidizing agent for bleaching compositions and from about 1.5 to about 15 percent by weight, and even more preferably from about 1.5 to about 10 percent by weight, of oxidizing agent for dyeing compositions. For certain preferred embodiments in which bleaching and dyeing are carried out using a single composition, the compositions preferably comprise from about 4 to about 10 percent by weight of oxidizing agent and even more preferably from about 5 to about 9 percent by weight of oxidizing agent.

The oxidizing agent to be used in accordance with the present invention may comprise any of a number of conventional or unconventional oxidizing agents. Generally, it is only required that the oxidizing agent be nontoxic, mild in action and free of harmful residue. For the removal of color from the hair, such as in bleaching operations, it is preferred that the oxidizing agent comprise a salt of persulfuric acid (H₂S₂O₈), and preferably alkali metal and ammonium salts of persulfuric acid. It is especially preferred for bleaching operations that the oxidizing agent be selected from the group consisting of sodium persulfate (Na₂S₂O₈), potassium persulfate (K₂S₂O₈), ammonium persulfate ((NH₄)S₂O₈), and mixtures of two or more of the these. Other suitable oxidizing agents, such as peroxides, for use in bleaching operations, in addition to those exemplified above, will be apparent in view of the present disclosure.

According to other embodiments of the present invention, the hair coloring composition comprises a dyeing agent for adding color to the hair. It is contemplated that the types of dyeing agent suitable for use in accordance with the present invention are numerous and varied, including temporary, semi-permanent and permanent dyes generally known to those skilled in the art. Examples of semi-permanent dyes include, for example, azo and nitro compounds and derivatives of naphthalene and anthraquinone. Such dyeing agents are generally referred to as “non-oxidative” dyeing agents, since these dyeing agents do not require oxidation to color the hair.

It is generally preferred, however, that the dyeing agent of the present invention comprise an oxidative dyeing agent. As the term is used herein, oxidative dyeing agent refers to those dye intermediates or precursors, which produce color upon oxidation. The oxidative dyeing agents of the present invention preferably comprise monomeric aromatic compounds, which, on oxidation, form oligomers or polymers having extended conjugated systems of electrons in their molecular structure. This oxidative reaction produces oligomers and polymers with electronic structures in the visible spectrum. As a result, oxidation of the dye intermediates or precursors results in the development of color. Especially preferred oxidative dying agents comprise substituted phenols, amino phenols, diamines, including the o-and p-diamines, aminohydroxy compounds of benzene, and derivatives of these which pass through a quinoid stage during oxidation. According to certain embodiments, aromatic amines having two functional groups, such as p-phenylenediamine, are preferred for their ability to yield higher molecular weight colored materials upon oxidative polymerization. Mono functionalized aromatic amines capable of yielding colored conjugated imines, and quinoid dimers, trimers, etc. are preferred according to other embodiments. It is also contemplated that combinations of these various dye precursors may be used.

Suitable oxidative dyeing agents to be used in accordance with the present invention are disclosed, for example, in U.S. Pat. Nos. 4,473,375 and 4,840,639 and F. E. Wall, “Bleaches, Hair Colorings, and Dye Removers,” Cosmetics: Science and Technology, Vol. 2, pp. 300-320 (1972). These references are incorporated herein by reference. Other dyeing agents to be used in the present invention would be readily apparent to one skilled in the art.

For embodiments comprising an oxidative dyeing agent, its highly preferred that the compositions include an oxidizing agent comprising hydrogen peroxide. It will be appreciated by those skilled in the art that such oxidizing agents operate to develop the color of the dye intermediate and that other developing agents, either alone or in combination with the present oxidizing agents, may be used within the scope of the present invention. What is required in such embodiments is that the developing agent, which is preferably an oxidizing agent, converts the synthetic organic hair coloring intermediates to the desired color.

Applicants have found that the present invention provides a surprising and beneficial result with respect to the amount of oxidizing agent required to develop the color of commonly used oxidative dyeing agents. It has heretofore been common practice to use a 6 wt. % aqueous solution of hydrogen peroxide for developing dye intermediates. Furthermore, it has also been common practice to employ about 2 parts by volume of such hydrogen peroxide solution for every one part by volume of dye base (i.e., dyeing agent plus carrier). In contrast, certain embodiments of the present invention utilize as little as 0.5 parts by volume of hydrogen peroxide solution (6 wt. %) for each part by volume of dye base without any noticeable decrease in hair color alteration. In fact, applicants have discovered that even with this reduced ratio, hair color alteration is effected in substantially shorter time periods than those required by the prior art.

2. The Coloring Agents—Carrier

It will be appreciated by those skilled in the art that many of the active compounds described herein are most readily available in the form of dispersions or solutions of one or more liquids, typically aqueous solutions. It is contemplated the active ingredients of the present invention will frequently be utilized in this from, and accordingly the present compositions preferably include a carrier for the coloring agent. In general it is contemplated that a wide variety of materials will be suitable for use as a carrier, and all such materials are within the scope of the present invention. It is generally preferred that the carrier comprise a liquid, preferably a polar liquid, for facilitating delivery and application of the present coloring agent to the hair. As will be appreciated by those skilled in the art, the physical condition of the carrier may therefore vary widely, ranging, for example, from a thin clear liquid to a creamy paste, depending upon the needs of the particular application.

The carrier of the present invention preferably comprises a solvent for one or more of the active components of the coloring agent. Thus, for compositions containing polar oxidizing agent and/or polar dyeing agents, the carrier preferably comprises a polar liquid, such as water, alcohol and mixtures of these. The term solvent is used in this context in a broad sense to include those liquid components and mixtures of liquid components, which have at least some tendency to solubilize at least one active component of the coloring agent. It is especially preferred that the carrier comprise a mixture of water and an alcohol and/or glycol, such as C₂-C₆ alcohols, preferably isopropyl alcohol, and C₂-C₆ glycols, preferably propylene glycol and/or hexylene glycol. The composition preferably comprises solvent in an amount from about 40 to about 85 percent by weight of the composition.

In accordance with a preferred embodiment of the present invention, the carrier comprises a thickening agent for adjusting the rheology of the composition. The type and amount of such thickening agents may vary widely within the scope of the present invention. Thus, the thickening agents suitable for use in the present compositions are those thickening agents typically used in cosmetics and generally include organic and inorganic compounds. Examples of suitable thickening agents include silica, carboxymethylcellulose, fatty alcohols and mixtures of two or more of these. It is preferred that the fatty alcohol comprises lauryl alcohol. A suitable lauryl alcohol is CO-1214, commercially available from Procter & Gamble of Cincinnati, Ohio. A suitable carboxymethylcellulose is CMC-7H3SF, commercially available from Hercules, Inc. of Wilmington, Del. The carrier preferably comprises an amount of the thickening agent to provide the aqueous composition with the desired thickness or viscosity. It is generally preferred, however, that the present coloring composition comprises from about 5 to about 15 percent by weight of thickening agent, with about 8 to about 12 weight percent being even more preferred in certain embodiments.

For embodiments in which the composition includes a dyeing agent, and particularly an oxidative dyeing agent, the carrier also preferably comprises an alkalizer or buffer for providing the proper environment for the dye intermediate, as is well known in the art. In certain embodiments the carrier preferably comprise an aqueous solution, and preferably a 28 wt. % solution, of ammonium hydroxide as alkalizer. In other embodiments, the alkalizer comprises sodium borate and/or urea. The amount of alkalizer will of course depend upon the particular dyeing agent and other factors. It is generally preferred, however, that the compositions of the present invention include from about 5 to about 10 percent by weight of 28 wt. % aqueous ammonium hydroxide solution, or from about 0.5 to about 5 percent by weight of urea (GR prilled from EM Science).

Embodiments including dyeing agent also preferably include spreading agent to assist in distribution of the dyeing agent evenly along the hair shaft. Suitable spreading agents include most well known surfactants, such as ethoxylated alkylphenols, and preferably octylphenoxy poly(ethyleneoxy)ethanol, which is commercially available as Igepal CA-630 from Rhone-Poulenc/GAF of Wayne, N.J. Spreading agents in form of polyethylene glycol octophenol ether (sold under the trade name TRITON X-100) are also preferred in certain embodiments.

The carrier also preferably comprises a detergent for leaving the hair feeling smooth and soft after treatment with the compositions of the present invention. Suitable detergents include those detergents readily known to those skilled in the art, including primary alkyl sulfates of the C12-C18 series, salts of oleic acid, ammonium hydroxide, zwitterionic compounds and mixtures of two or more of these. Preferably, the detergent comprises ammonium lauryl sulfate. A suitable ammonium lauryl sulfate is Emersal 633LL.sup.R, commercially available from Emery Chemicals Personal Care and Specialties Group of Linden, N.J. Examples of oleic acid and aqueous ammonium hydroxide which are readily available include Emersal 6333LL.sup.R, commercially available from Emery Industries, Inc., Fatty and Dibasic Acids Group, Cincinnati, Ohio. An example of a suitable zwitterionic compound to be used in accordance with the present invention is lauramido propyl betaine, commercially available from Mona Industries, Inc. of Patterson, N.J. Other detergents suitable for use in the carrier of the aqueous solution of the present invention would be readily apparent based upon the present disclosure.

In a preferred embodiment of the present invention, the carrier comprises chelating agent. The purpose of the chelating agent in the present compositions is to chelate or bind heavy metals, which may be present in the water of the aqueous compositions. In the absence of such chelating agent, such metal ions may deleteriously affect the performance of the active color components. Accordingly, the amount and type of chelating agent will depend, for example, on the quality of the water used in the carrier and the sensitivity of the active color components. Thus, the chelating agent may comprise any of a number of conventional or unconventional chelating agents used in conventional amounts. It is preferred, however, that the chelating agent comprise, and preferably consist of, ethylenediaminetetraacetic acid (“EDTA”). An example of a suitable EDTA to be used in accordance with the present invention is Hamp-ene acid® commercially available from W. R. Grace and Co. of Nashua, N.H. For embodiments in which the coloring agent comprises oxidative dyeing agent, it is generally preferred that the carrier include an anti-oxidant to assist in the prevention of premature decomposition of the dye intermediates. It is contemplated that customary types and amounts of anti-oxidants may be used within the scope of the present invention. Sodium sulfite and ascorbic acid are antioxidants, which may be used, in customary amounts in the compositions of the present invention.

B. The Maintaining Agent

An important aspect of the present invention is the provision of hair coloring compositions containing both any reducing saccharide and a non-reducing saccharide. Those skilled in the art will appreciate that the term “maintaining agent” as used herein is intended to encompass not only compositions in which the two components are mixed together and then incorporated into a hair treatment composition, but also hair treatment compositions in which the reducing saccharide and the non-reducing saccharide are incorporated at separate times into the hair treatment composition.

In addition to having the synergistic and unexpected results it is contemplated that the reducing saccharide of the present invention acts as a protecting agent for protecting the keratin fibers of the hair from unfavorable damage and degradation while also permitting, and preferably enhancing, the oxidation of melanin contained in the hair. Furthermore, applicants have found that the presence of non-reducing saccharide in the present hair treatment compositions tends to also protect the scalp of the person being treated and the hands of the hair care professional from irritation and burning. In embodiments in which the composition includes oxidative dyeing agent, the reducing saccharide appears to also act as a protecting agent to the extent that it inhibits premature or excessive development of the dye intermediate.

An especially preferred embodiment of the present invention provides coloring compositions in which the coloring agent comprises oxidative dyeing agent and oxidizing agent, the amount and type of the oxidizing agent being effective to develop said oxidative dyeing agent and to remove color from the hair. Such embodiments are preferred for the advantage of providing a composition, which is highly effective for substantially simultaneously removing and adding color to hair, a characteristic that has been long sought but not heretofore fully achieved. The difficulty encountered by prior art compositions stemmed from the conflicting and contradictory requirements of the components of such compositions. In particular, bleaching of hair to remove color has heretofore generally required a type and amount of oxidizing agent, which has been detrimental to effective performance of the dye intermediates. Thus, it has heretofore been difficult if not impossible to effectively formulate compositions containing a type and amount of oxidizing agent effective to perform both lightening and development functions. Applicants have unexpectedly found that such a characteristic is possessed by certain preferred embodiments of the present compositions.

Without being bound by or limited to any particular theory of operation, it is believed that the ability of the present composition to both lighten hair and develop dye intermediates is due, at least in part, to the beneficial effects of reducing saccharide in the present compositions. In particular, it is believed that the reducing saccharide component, and preferably glucose, maltose and combinations of these two, favorably regulate development of the dye intermediate, enhances the oxidative reaction of the melanin in the hair, and moderates harmful oxidative attack on the keratin fibers in the hair. In connection with regulation of the reaction of the dye intermediates, it is contemplated that the reducing saccharide may interfere to a favorable extent with the chemical interaction of the oxidizing agent and the dye intermediate. Furthermore, it is believed that, in the absence of reducing saccharide, bleaching of hair by exposure to oxidizing agents causes a disadvantageous degradation of the keratin fibers, and that this degradation inhibits binding of the developed dye to the keratin fiber. According the present invention, it is expected that the reducing saccharide acts to preferentially favor reaction of the oxidizing agent with the melanin while simultaneously protecting the keratin fibers from degradation. Applicants have thus discovered that the inclusion of reducing saccharide, such as glucose, in compositions for the coloring of hair imparts several desirable characteristics to such compositions.

As is well known to those skilled in the art, reducing disaccharides are carbohydrates comprised of two monosaccharide units. As used herein, the term reducing saccharide refers to all known and available reducing saccharide compounds, including all stereoisomeric and enantiomeric forms thereof. While it is contemplated that all such reducing saccharides are adaptable for use in the compositions of the present invention, it is highly preferred that the reducing saccharide comprise, and preferably consist essentially of D-Glucose for embodiments involving the use of active dying agents and the non-reducing saccharide comprises, and preferably consist essentially of, sucrose. For embodiments involving the use of bleaching without necessarily dying, it is highly preferred that the reducing saccharide comprise, and preferably consist essentially of, maltose, and even more preferably maltose monohydrate. It is also preferred that the reducing saccharide of the present invention reduces Tollens' or Fehlings' reagent.

It is contemplated that the amount of reducing saccharide used in accordance with the present invention may vary widely, depending upon numerous factors, such as hair type and the desired color alteration. It is generally preferred, however, that the coloring compositions of the present invention comprise from about 0.1 to about 5 percent by weight of reducing saccharide, with from about 0.5 to about 3 percent by weight being more preferred, and 0.1 to about 2 percent being even more preferred according to certain embodiments. According to especially preferred embodiments in which the bleaching and dyeing agents are brought together at the time of use to form a composition in accordance with the present invention, the coloring compositions comprise about 1 to about 2 percent by weight of reducing saccharide.

As mentioned hereinbefore, the ratio of reducing saccharide to active color compounds is an important aspect of certain embodiments of the present invention. It is generally preferred that such ratio, on a weight basis, is from about 0.01:1 to about 0.1:10, and even more preferably from about 0.01:1 to about 0.1:1. For coloring compositions which do not contain oxidative dyeing agents, for example bleaching compositions, the reducing dissaccharide:active coloring compound weight ratio is preferably from about 0.1:1 to about 0.3:1. For coloring compositions, which do contain oxidative dyeing agents, the weight ratio of reducing saccharide: active coloring compound is preferably from about 0.02:1 to about 0.04:1.

C. Coloring Compositions—The Kits

According to certain embodiments of the invention, the present compositions are provided in kit form and preferably comprise a first container containing a first composition for adding color to the hair, a second container containing a second composition comprising carrier components and a third container containing an oxidizing agent. In such embodiments, it is contemplated that reducing saccharide is preferably included in either the first composition or the second composition or both the first and second compositions. It is contemplated that the hair care professional or in-home user can combine the contents of the kit to produce numerous and varied compositions of the present invention, thereby having the ability to achieve a range of hair coloring effects.

The first container preferably contains a composition comprising dyeing agent, and even more preferably a composition comprising oxidative dyeing agent. For the purpose of convenience, first compositions, which contain dyeing agent, are sometimes referred to herein as dye base. It is preferred that the first composition of the kit of the present invention is itself a composition according to the present invention. That is, it is preferred that the first composition also contain reducing saccharide. In such embodiments, the composition in the first container preferably comprises from about 0.2 to about 5 percent by weight of oxidative dyeing agent, from about 1 to about 5 percent by weight of reducing saccharide and about 85 to about 95 percent by weight of carrier.

The second composition, which is also preferably a composition of the present invention comprising carrier components and reducing saccharide, is sometimes referred to herein as bleach oil. The bleach oil preferably consists essentially of carrier components and reducing saccharide wherein the amount of reducing saccharide is preferably from about 3 to 5 percent by weight of the bleach oil. Other ingredients adaptable for use in the present compositions to affect the handling, rheology, etc., as described hereinbefore, may also be included in the composition in the second container.

The third container contains a composition, and preferably a composition in solid powder form, comprising oxidizing agent. The oxidizing agent is preferably selected so as to remove color from the hair. Thus, for the purpose of convenience, the third composition is sometimes referred to herein as bleach booster powder. For example, the composition contained in the third container preferably includes oxidizing agent comprising a salt of persulfuric acid (H₂S₂O₈), and preferably alkali metal and ammonium salts of persulfuric acid, such as sodium persulfate (Na₂S₂O₈), potassium persulfate (K₂S₂O₈), ammonium persulfate ((NH₄)₂S₂O₈). Other ingredients adaptable for use in the present compositions, as described hereinbefore, may also be included in the composition of the third container.

In operation, the bleach oil is adapted to be combined with a portion of the contents of the first container, or with a portion of the contents of the third container, or with a portion of the contents of both the first and third containers to produce a composition according to the present invention. For example, the oxidizing agent in the third container is especially adapted to remove color from hair when combined with bleach oil described above. Thus, a bleaching composition according to the present invention is prepared by mixing a portion of the first and third containers hereof. Furthermore, the composition in the first container is suitable for use alone or in combination with a peroxide solution to add color to hair. For embodiments in which both removal of color from and addition of color to the hair is desired, portions of the contents of all three containers may be combined.

In all cases, it is contemplated that an aqueous peroxide solution, and preferably a 20-vol solution, which is normally not part of the kit, will preferably be utilized to produce a final composition according to the present invention.

III. Coloring Methods

The present invention provides methods for conveniently coloring hair comprising applying a composition of the present invention to the hair, allowing said composition to remain in contact with the hair for a time sufficient to alter the color of the hair and substantially removing said composition from the hair.

According to especially preferred aspects of the present invention, the methods achieve bleaching and dyeing of hair in a one-step procedure. This method comprises applying to the hair an aqueous hair coloring composition comprising an oxidative dyeing agent, reducing saccharide, and oxidizing agent, the amount and type of the oxidizing agent being effective to develop said oxidative dyeing agent and to remove color from the hair. Such methods are highly preferred for the ability to achieve effective removal of color from hair and addition of color to hair in a single step. The composition is preferably allowed to remain in contact with the hair for a time sufficient to bleach and dye the hair to the desired degree. The composition is then removed from the hair, preferably by steps, which comprise rinsing with water.

The application of the compositions of the present invention to the hair comprises application procedures generally known to those skilled in the art. For example, the hair may be dry upon application of the compositions. Conversely, the hair may be wetted with water prior to the application of the compositions. In either event, the composition is applied in an amount so that it is substantially completely and evenly distributed throughout the hair.

In accordance with the method aspects of the present invention, the composition is allowed to remain in contact with the hair for a period of time effective to color the hair by both bleaching and dyeing the hair. Preferably, the composition is allowed to remain in contact with the hair for about 5 to about 20 minutes. Applicants have found that, due in large part to the beneficial properties of the present compositions, the contact times required by the preferred methods of the present invention to achieve a desired color alteration are substantially shorter than those required by prior art procedures. Accordingly, the color altering compositions of the present invention are preferably allowed to remain in contact with the hair for no more than about 10 minutes, and even more preferably from about 5 to about 10 minutes.

After the color of the hair has been altered to the desired extent, the compositions of the present invention are preferably removed from the hair by rinsing the hair with water. Thereafter, the hair may be cut, styled and dried in any desirable manner.

In accordance with the present invention, maintaining agents are used in combination with various caustic hair treating compositions to protect the hair and scalp from the effects of the caustic component, to improve the performance of the composition and/or to reduce the odor of the composition.

IV. Shaping Compositions

U.S. Pat. No. 5,639,451 discloses a shaping composition for increasing the malleability of hair comprising an aqueous solution containing cysteine, cysteamine and sucrose, which is a non-reducing reducing saccharide, and having a pH in the range of about 5 to about 13. It was found that the inclusion of cysteamine in compositions according to the teachings of this patent provided compositions with highly desirable and unexpected properties. In particular, the compositions of this patent were found to be non-damaging to the hair and, at the same time, fast-acting and free of unpleasant odor, a combination of properties not theretofore achieved in such hair care products containing cysteamine. Furthermore, the compositions have also been found to produce shaped hair with exceptional feel and texture and with little or no embrittlement or damage.

However, the present inventors have found that when such compositions in accordance with the '451 patent are caustic, with a pH in excess of about 7, that the compositions may be irritating to the scalp of the subject undergoing treatment. In such instances, efforts may need to be taken to protect the scalp from prolonged exposure to the hair treating compositions. Such efforts may include applying a separate protective composition to the scalp prior to treatment, physically masking the scalp from the hair treatment composition, or limiting the exposure time to the composition. Applying a protective composition or physical mask to the scalp entails extra steps, and may not be fully effective in protecting the scalp. Reducing treatment time may reduce the effectiveness of the hair treatment.

In accordance with the present invention, it has been found that the addition of maintaining agent to the hair treatment compositions reduces the scalp irritating effects of the caustic compositions while also providing the favorable effects of the sucrose as discussed in the '451 patent.

Certain of the shaping compositions of the present invention comprise (a) from about 0.3 to about 11% by weight cysteine; (b) from about 2 to about 15% by weight cysteamine; and from about 0.1 to about to about 12% by weight maintaining agent in a caustic aqueous solution having a pH of about 6.8 to about 9.2, preferably at least about 8.3.

A. The Shaping Agents

The hair shaping compositions of the present invention contain a shaping agent. The term shaping agent is used herein in a non-limiting sense to refer to any agent, compound or composition adapted to permanently alter the shape of hair. Thus, the shaping agents of the present invention include those compositions, which tend to straighten naturally curly or kinky hair as well as those, which tend to curl naturally straight hair. Furthermore, the term shaping agent is intended to include reducing agents, which break cystine disulfide bonds in hair as well as oxidizing agents, which establish such disulphide bonds in hair. For the purpose of convenience, shaping compositions, which tend to cause breaking of the bonds, which contribute to the shape of the hair, such as cysteine disulphide bonds, are sometimes referred to herein as softening compositions. On the other hand, shaping compositions, which tend to establish or reestablish such bonds are sometimes referred to herein as fixing compositions.

It is contemplated that the shaping agents of the present invention will generally include one or more active shaping compounds and carrier for the active compounds. It will be appreciated that the terms active shaping compound and carrier are used herein for the purpose of convenience and illustration but not by way of limitation. In particular, the term active shaping compound refers to those components of the shaping agent, which interact chemically with the hair to alter the shape of the hair. In contrast, the carrier serves principally to provide the proper environment for the active compounds and to facilitate, enhance and/or modify delivery and application of the active compounds to the hair. The preferred shaping agents of the present invention comprise from about 10 to about 25 percent by weight of active shaping compound plus cysteamine. The shape altering compositions preferably include from about 3 to about 20 weight percent maintaining agent and from about 55 to about 87 percent by weight of carrier.

An important aspect of the present invention resides in the weight ratio between the active shaping compound and the other two essential ingredients of the composition: the cysteamine and the maintaining agent. Considering first the ratio of maintaining agent to active shaping compounds and cysteamine, it is believed that such ratio may vary widely, depending upon numerous factors, such as the type of hair and the degree of shape alteration desired. It is preferred, however, that the weight ratio of the maintaining agent to each of the active shaping compound and the cysteamine be from about 0.1:1 to about 4:1. As regards the weight ratio of the active shaping compound and the cysteamine, these are generally between 4:1 and 1:4.

B. Active Shaping Compounds

According to certain embodiments of the present invention, the active shaping compound comprises a reducing compound. As will be appreciated by those skilled in the art, reducing compounds are commonly used as active components for cleaving or breaking the cystine disulphide bonds in hair. Thus, as the term is used herein, reducing compound refers to those compounds, which are reducing agents with respect to cystine in the hair. The amount of reducing compound contained in the present compositions will vary widely, depending upon the particular circumstances of each embodiment. It is generally preferred, however, that the present compositions comprises from about 5 to about 20 percent by weight of reducing compound.

The reducing compound to be used in accordance with the present invention may comprise any of a number of reducing compounds conventionally used for the purpose of waving or straightening hair. Generally, it is only required that the reducing compound be nontoxic and free of harmful residue. Thus, the reducing compound of the present invention is preferably selected from the group consisting of sulfites, mercaptans, e.g., thioglycolic acid and the salts thereof, thiolactic acid, monothioglycerol, 1-thioglycerol, 3-mercapto-propionic acid, including cysteine, and mixtures of two or more of the foregoing. Other suitable reducing compounds for use in shaping compositions, in addition to those exemplified above will be apparent in view of the present disclosure.

According to a presently preferred embodiment of the invention, it is preferred to use cysteine as the active shaping compound for reductively cleaving the sulfur-sulfur bonds of the cystine bridges in hair. It has been found that such cleavage tends to “soften” the hair. As the term is used herein, “softened hair” refers to hair, which has been rendered malleable relative to its natural resiliency. In the compositions of the present invention, the active shaping compound, such as cysteine, is believed to cause reductive cleavage of at least a portion of the disulfide bonds within and between the individual protein chains which comprise the hair. The resulting softening of the hair allows the hair to be more readily reconfigured.

Cysteine, 2-amino-3-mercaptopropionic acid, has the empirical chemical formula C₃H₇NO₂S and the molecular chemical formula given below: 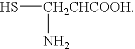
It will be understood that the term cysteine as used herein encompasses within its scope all the various enantiomeric and ionic forms that the cysteine molecule is capable of taking in solution, it being contemplated that all such forms will be capable of performing the desired function. For example, unless otherwise specifically designated herein, the term cysteine includes both L and D enantiomers of that component. It is believed that, up to the solubility limit of cysteine, all concentrations of cysteine in the softening compositions of the present invention will have a degree of effectiveness and therefore all such concentrations are within the broad scope of the present invention. It is preferred, however, that the concentration of cysteine range from about 5 weight percent to about 20 weight percent, and more preferably from about 6 weight percent to about 14 weight percent, depending upon the pH of the solution. For example, when the solution has a pH of between about 8 and about 10, it is preferred that the cysteine concentration range from about 13 weight percent to about 14 weight percent.

According to many preferred embodiments, hair shaping is advantageously carried out under alkaline conditions. Thus, the shaping agent of the present invention preferably includes an alkalizing agent. Such alkalizing agents may be used alone or in combination with the reducing agent or oxidizing agent of the present invention. Alkalizing agents adaptable for use in hair shaping operations are well known in the art, and all such alkalizing agents are within the scope hereof. It is generally preferred, however, that the alkalizing agent of the present invention be selected from the group consisting of hydroxides, carbonates and mixtures of these. Especially preferred are the sodium, potassium and ammonium hydroxides and carbonates.

According to other embodiments of the present invention, the active hair-shaping compound comprises an oxidizing compound for creating cross-linkages between adjacent peptide chains of the keratin in the hair, and especially for restoring cystine disulphide bonds in hair. It is contemplated that the types of oxidizing compounds suitable for use in accordance with the present invention are numerous and varied, and all such oxidizing compounds are within the scope hereof. It is generally preferred, however, that the oxidizing compound of the present invention be selected from the group consisting of peroxides, perborates, bromates and mixtures of two or more of the aforementioned. Hydrogen peroxide is an especially preferred oxidizing compound due to its low cost, mild action and ready availability.

It is contemplated that other agents for nonoxidative crosslinking of adjacent peptide chains may also be beneficial in certain embodiments of the present invention. For example, it is contemplated that the shaping agent may comprise a nonoxidative crosslinking agent selected from the group consisting of bivalent metal ions, alkylene dihalides, and aldehydes.

C. The Carrier

It will be appreciated by those skilled in the art that many of the active compounds described herein are readily available in the form of solutions or suspensions of one or more liquids, typically aqueous solutions. It is contemplated that the active compounds of the present invention will frequently be utilized in this form, and accordingly the present compositions preferably include a carrier for the active shaping compound. In general it is contemplated that a wide variety of materials will be suitable for use as a carrier, and all such materials are within the scope of the present invention. It is generally preferred that the carrier comprise a liquid, preferably a polar liquid, for facilitating delivery and application of the present shaping agent to the hair. As will be appreciated by those skilled in the art, the physical condition of the carrier may therefore vary widely, ranging, for example, from a thin clear liquid to a creamy paste, depending upon the needs of the particular application. The carrier of the present invention preferably comprises a solvent for one or more of the active components of the shaping agent. Thus, for compositions containing polar shaping compounds, the carrier preferably comprises a polar liquid, such as water, alcohol and mixtures of these. The term solvent is used in this context in a broad sense to include those liquid components and mixtures of liquid components, which have at least some tendency to solubilize at least one active component of the shaping agent. It is especially preferred that the carrier comprise a mixture of water and an alcohol. The composition preferably comprises solvent in an amount from about 55 to about 87 percent by weight of the composition.

According to certain preferred embodiments, such as compositions especially adapted for the straightening of kinky or curly hair, the carrier comprises an oil-in-water emulsion having a continuous water phase and a disperse oil phase. In such embodiments, the active shaping compound is maintained in the form of a solution or suspension in the water phase of the carrier. Such oil-in-water emulsions are preferably relatively highly viscous, creamy materials. These properties and characteristics assist in the application the compositions to the hair. Furthermore, the rheology of these compositions tends to hold the hair in a straightened configuration during processing. Compositions of this type are preferred when the active compound comprises alkali in major proportion. In such embodiments, it is contemplated that the carrier will also include emulsifying agents to aid in the formation and maintenance of such emulsions.

In accordance with a preferred embodiment of the present invention, the carrier comprises a thickening agent for adjusting the rheology of the composition. As the term is used herein, a thickening agent includes any agent which provides a high viscosity to the softening solution thus making it easier to apply. Moreover, the relatively high viscosity imparted by the thickening agent enhances uniform spreading of the treating solution and retards dripping and evaporation of the solution. According to certain embodiments, it is preferred that the thickening agent be water-soluble so that it may be readily included in the aqueous solutions of the present invention. The thickening agents suitable for use in the present compositions are those thickening agents typically used in cosmetics and generally include organic and inorganic compounds. Examples of suitable thickening agents include: silica; carboxy alkylcellulose, such as carboxymethylcellulose; fatty alcohols; mineral oils; gelatins; hydroxy alkylcellulose, such as hydroxy ethylcellulose; and mixtures of two or more of these. The concentration of the thickening agents in the compositions of the present invention will vary greatly according to the desired result in each individual case, and, accordingly, all such concentrations are within the scope of the present invention. Applicant has found, however, that thickening agent concentrations of from about 0.5 weight percent to about 2.0 weight percent are preferred for certain embodiments.

The carrier also preferably comprises a detergent and/or conditioner for leaving the hair feeling smooth and soft after treatment with the compositions of the present invention. Suitable detergents include those detergents readily known to those skilled in the hair care art, including primary alkyl sulfates of the C₁₂-C₁₈ series, salts of oleic acid, ammonium hydroxide, zwitterionic compounds and mixtures of two or more of these. Other detergents suitable for use in the carrier of the aqueous solution of the present invention would be readily apparent based upon the present disclosure.

According to certain preferred embodiments, the compositions of the present invention further include at least one penetrating agent. As the term is used herein, a penetrating agent is any material, which improves penetration of the solutions into the hair. Although any penetrating agent heretofore used for the purpose can be used in the present invention as well, particularly preferred penetrating agents are propylene glycol, monoethanolamine, any compound containing an ethoxylate group, and oleth-20, the latter being the generic term for the polyethyleneglycol ether of oleyl or laural alcohol having the formula: CH₃(CH₂)₇CH═CH(CH₂)₇CH₂(OCH₂CH₂)ₙOH, wherein n has an average value of about 20. Oleth-20 is sold under the trade name “BRIJ” by ICI Americas Corp. The concentration of the penetrating agents to be used according to the compositions of the present invention will vary greatly depending upon the amount of penetration desired, and, accordingly, all such concentrations are within the scope of the present invention. Applicant has found that compositions containing a penetrating agent in concentrations from about 4 weight percent to about 10 weight percent are preferred according to certain embodiments.

In certain embodiments, it is preferred that the compositions of the present invention include chelating agents. As the term is used herein, a chelating agent is any polydentate ligand capable of forming a complex with a metal ion. While many such chelating agents are readily available and well known in the art, common chelating agents include amine containing acids, hydroxy carboxylic acids, dicarboxylic acids, alkali metal salts of the foregoing acids, and mixtures of these. It is preferred that the chelating agent comprise hydroxy ethylenediaminetriacetic acid (hereinafter H-EDTA) and even more preferably alkali metal salts of H-EDTA. It is generally preferred that sodium salts of H-EDTA be used as the chelating agents in the compositions of the present invention, and it is even more preferred that trisodium salt of H-EDTA is used as the chelating agent. The inclusion of chelating agents in the compositions of the present invention beneficially removes toxic heavy metals, such as mercury, cadmium, and lead, which otherwise bind to the thio groups of cysteine in the hair protein. Freeing these thio groups is beneficial since the freed thio groups aid in forming the new disulfide linkages after the hair has been reconfigured, thereby helping to maintain the hair in its new configuration. The concentration of the chelating agents in the compositions of the present invention will vary greatly depending upon the circumstances of use in each individual case, and, accordingly, all such concentrations are within the scope of the present invention. Applicant has found however, that chelating agents are preferably included in the compositions of the present invention in concentrations from about 0.25 weight percent to about 1.5 weight percent.

D. Cysteamine Component

Cysteamine, 2-aminoethyl mercaptan (decarboxycysteine), has the empirical chemical formula C₂H₇NS and the following molecular chemical formula:
HS—CH₂CH₂—NH₂.

In the compositions of the present invention, it behaves as a supplemental shaping agent, providing compositions having faster fixing activity than do similar shaping compositions that do not contain cysteamine. Surprisingly, however, the cysteamine containing shaping compositions of the present invention do not cause the offensive odor associated with previously used shaping compositions that contain cysteamine.

In particular, it has been discovered that when cysteamine is incorporated into the shaping compositions of the present invention, in an amount of from about 5 weight percent to about 20 weight percent of the total composition, the shaping composition has unexpectedly improved shaping properties, particularly with respect to speed of action, without the unpleasant odor heretofore associated with the use of cysteamine in shaping compositions. This is particularly true when the composition comprises a total of from about 10 weight percent to about 25 weight percent of active shaping compound plus cysteamine and from about 3 weight percent to about 20 weight percent of maintaining agent.

E. The Maintaining Agent

An important aspect of the present invention is the provision of hair altering compositions containing a maintaining agent. In particular, it is contemplated that the saccharides of the present invention acts as an anti-oxidant, and thus protect the hair and scalp from oxidation. The reducing sugar also helps promote the activity of the cysteine and cysteamine components, when present, which act by a reducing process. Furthermore, the reducing sugars help to control unpleasant smells by reducing the production of di-thiol side products, which may be formed. Thus the reducing sugars accelerate the activity of the reducing agents in the hair shaping compositions, reduce oxidation of these agents both in the bottle and in use, and prevent surface oxidation of hair and scalp during use.

The active reducing agents in the hair shaping compositions may comprises cysteine and cysteamine. Other hair treatment compositions also have active reducing agents. The bleach oil and hair color base compositions of the present invention preferably include reducing agents, such as sodium sulfite and/or trisodium phosphate. In the relaxer composition of the present invention, the reducing agent is the sodium hydroxide. In each case the reducing sugars promote the activity of these reducing agent components, while reducing undesirable oxidation in the bottle and in use, and protecting the hair and scalp from surface oxidation.

As discussed in the '451 patent, the sugars also acts as a protecting agent for protecting the keratin fibers of the hair from undesirable damage and degradation while also permitting, and preferably enhancing, the softening of the hair. Furthermore, the reducing saccharide also protects the scalp of the person being treated and the hands of the hair care professional from irritation and burning. While applicants do not intend to be bound by or limited to any particular theory, it is believed that the reducing saccharide provides abundant sites for hydrogen bonding with the molecules which make up the hair. The availability of such sites is believed to compete for the otherwise intermolecular hydrogen bonding which is present among the protein strands. This in turn opens the tertiary or spatial structure of the protein fibers, thereby facilitating reduction of the disulfide, cystine, down the thiol, cysteine.

As is well known to those skilled in the art, saccharides are carbohydrates and as used herein the term saccharide refers to all known and available saccharide compounds, including all stereoisomeric and antiomeric forms thereof. The reducing saccharides of the present invention are characterized as “reducing” because they reduce Tollens' or Fehlings' reagents, as is well known in the art. Preferred reducing saccharides include the disaccharides maltose and lactose, and the monosaccharide glucose. Maltose has been found particularly preferable for use in connection with the compositions of the present invention. According to preferred embodiments, the reducing saccharide comprises, and preferably consists essentially of, D-(+)-glucose. Glucose products adaptable for use in accordance with the present invention are available from Corn Products International under as a family of products sold under the trade designation Cerelose® dextrose, and all such products are considered to be adaptable for use in the preferred embodiments of the present invention, with monohydrate forms being generally preferred.

It is contemplated that the amount of reducing saccharide in the compositions of the present invention may vary widely, depending upon numerous factors, such as hair type and the desired shape or color alteration. It is generally preferred, however, as indicated above, that the shaping composition of the present invention comprise from about 0.1 to about 12 percent by weight of reducing saccharide, with amounts of from about 0.1 to about 5 percent by weight being more preferred, and amounts of from about 1 to about 3 percent being even more preferred.

In accordance with the presently preferred embodiment, the ratios between the shaping compound, preferably cysteine, the cysteamine, and the polysaccharide in the shaping compositions of the present invention may vary widely, depending on the particular application, as long as the foregoing parameters (a minimum of 5 weight percent of each of cysteine and cysteamine, a total of from about 10 weight percent to about 25 weight percent of cysteine and cysteamine combined, and a total of from about 3 weight percent to about 20 weight percent of polysaccharide), are maintained.

As mentioned above, cysteine is generally very unstable in a solution and is readily oxidized by oxygen, which may be dissolved in the solution. This oxidation is manifest in the formation of solid precipitate when an aqueous solution of cysteine is allowed to stand for more than a few hours. Applicant has found that the inclusion of a reducing saccharide in an aqueous solution of cysteine stabilizes the solution and protects the cysteine from oxidization or other degradation and extends the shelf life of the product.

With respect to embodiments in which the active shaping compound comprises cysteine in major proportion, applicant believes that the inclusion of a maintaining agent in any measurable concentration in the composition of the present invention will be effective to a degree in stabilizing the softening solution. That is, applicant has found that the concentration of the saccharide impacts the stabilizing effect in degree only and therefore may be varied over a wide range as desired. In particular, those solutions having relatively low concentrations of saccharide will tend to provide a shorter shelf life than those solutions having relatively high saccharide concentrations. Moreover, high concentrations of saccharide in the solution may tend to make the softening composition syrupy and more viscous than is desired in certain embodiments. Accordingly, for embodiments in which the active shaping compound comprises cysteine in major proportion, all concentrations of maintaining agent are within the scope of the present invention. Applicant has found, however, that in such embodiments the concentration of maintaining agent preferably ranges from about 0.1 weight percent to about 11 weight percent, more preferably from about 4 weight percent to about 10 weight percent. Furthermore, for shaping compositions in which the active shaping compound comprises cysteine in major proportion, the active shaping compound:saccharide weight ratio is preferably from about 2:1 to about 8:1.

Applicants have found that solutions, preferably aqueous solutions, of cysteine and a maintaining agent are effective in reductively cleaving the sulfur-sulfur bonds of the cystine bridges in hair, and that such cleavage tends to “soften” the hair. As the term is used herein, “softened hair” refers to hair, which has been rendered malleable relative to its natural resiliency. In the compositions of the present invention, cysteine (as well as other active shaping compounds) and cysteamine are believed to cause reductive cleavage of at least a portion of the disulfide bonds within and between the individual protein chains, which comprise the hair. This cleavage softens the hair and allows the hair to be more readily reconfigured. Applicant has thus discovered a softening composition, in the form of an aqueous solution, which contains the natural and effective reducing agent cysteine in combination with the rapid acting reducing agent cysteamine and which has no offensive odor, a high degree of stability and a relatively long shelf life. It is believed that the inclusion of maintaining agents in accordance with the present invention promotes the reductive cleavage activity of the cysteine and cysteamine, thereby aiding in process and reducing the time needed for the hair treatment.

For embodiments of the present invention in which the active shaping compound comprises alkali in major proportion, the amount of maintaining agent present in the composition is important to fully achieving the objects of the present invention. In particular, it has been discovered that such compositions, in general, exhibit a gradually increasing effectiveness as the amount of maintaining agent in the composition is increased. Accordingly, for shaping compositions in which the active shaping compound comprises alkali, the active shaping compound:saccharide weight ratio is preferably from about 2:1 to about 10:1.

It is generally preferred that compositions of the present invention comprise an aqueous solution which is not highly caustic. In particular, it is preferred that the pH of the solution be from about 6.8 to about 9.2 for embodiments in which the active shaping compound comprises cysteine in major proportion. More preferably, the pH is in the range of from about 8.3 to about 9.2. Thus, in such preferred embodiments of the present invention, the pH of the reducing composition is high enough to help break the ionic bridges between adjacent protein molecules and thus to enhance the softening capacity of the composition but is low enough to be safe to the scalp and skin. According to one preferred embodiment of the present invention, an alkalizing agent such as aqueous ammonia (ammonium hydroxide) is present to adjust the pH of the composition as required. In certain preferred embodiments, 28% aqueous ammonia is present in a concentration of about 0.1 weight percent to about 10 weight percent. In an alternative embodiment, ethanolamine can be substituted in equivalent amounts for the ammonium hydroxide.

V. Shaping Methods

The present invention also provides methods for modifying the natural conformation of existing hair. As the term is used herein, existing hair refers to fully developed excutaneous hair. As used herein, the term “natural configuration” refers to the configuration of the hair prior to being treated according to the methods of the present invention. That is, the term “natural configuration” is used for convenience only and does not limit the methods of the present invention to treatment of hair which has not been previously treated. Surprisingly and beneficially, the use of the methods and compositions of the present invention provide the capability for effective and non-damaging treatment of previously treated hair. In contrast to the methods and compositions heretofore used, the present invention achieves softening or relaxation of the hair by relatively benign repositioning of the natural constituents of the hair without causing permanent damage thereto. As a result, hair may be subjected to a plurality of treatments without being damaged to any substantial extent.

An important aspect of the methods of the present invention is application of the compositions of the present invention to the hair of the person to be treated. While many methods are known and available to those skilled in the art for the application of softening solutions and those methods may be readily adapted for use with the present compositions, it is preferred that the present compositions be directly applied to the hair. In particular, it is preferred that the application start at the scalp and move progressively outward towards the ends of the hair until the hair being treated is covered thoroughly. It will be understood by those skilled in the art that the particular application method step described above provides unique and substantial advantages over those methods generally used in the prior art. For example, application of the highly alkaline softening compositions heretofore used generally presented a serious risk of caustic burn to both the subject being treated and the person conducting the treatment (hereinafter “the operator”). Accordingly, prior methods require that precautions be taken in order to protect both the user and operator from caustic burn. For example, kits containing such solutions generally include instructions recommending or requiring that the operator wear gloves during the application step and that the skin of the treated person be protected from the solution by thick and highly viscous gels. Due to the relatively benign nature of the present compositions, such cumbersome and inconvenient precautions are not necessary. Moreover, it is imperative according to the heretofore-used methods that the application period be strictly controlled and minimized so as to avoid damage to the scalp and hair of the person being treated. Overexposure of the hair to such highly caustic solutions would generally cause severe and irreversible degradation of the structure of the hair. In comparison, the benign nature constituents of the softening solutions according to the present invention eliminates the criticality of the application period and extends the maximum application period nearly indefinitely, especially for embodiments in which the active compound comprises cysteine in major proportion.

As will be appreciated by those skilled in the art, the amount of softening composition to be applied according to the present invention will vary greatly depending upon a host of individual circumstances. For example, the particular type of hair, which is to be treated, will have a large impact on the amount of composition to be applied. In particular, it is well known that different types of hair have varying degrees of moisture absorbency. Since softening compositions of the present invention preferably comprise an aqueous solution, the ability of the composition to effect hair softening will depend to some extent upon this property of the individual hair. Likewise, the amount of hair to be treated will also determine the application rate of the softening composition. In addition, the extent to which the natural configuration of the hair is to be modified will also impact upon the amount of the composition to be applied. Accordingly, all application rates and amounts are within the scope of the present invention. Applicant has found, however, that based primarily upon considerations of cost and convenience, that the amount of solution to be applied is preferably from about 3 ounces to about 4 ounces and more preferably from about 3.5 ounces to about 4 ounces.

The application period for the compositions of the present invention will also vary widely depending upon a variety of individual circumstances. Importantly and surprisingly, applicant has found that exposure of the hair and scalp to certain compositions of the present invention for extended periods of time will not result in any substantial degradation of the hair or cause deleterious effects to the scalp. On the contrary, it is believed that extended exposure of the hair to the compositions of the present invention, especially those compositions having a pH of about 7, may tend to invigorate and revitalize the hair rather than cause the degradation thereof.

While applicant does not intend to be bound by or to any particular theory, both cysteine and cystine are naturally occurring amino acids in keratin and therefore it is believed that extended exposure of the hair to compositions of the present invention comprising cysteine will tend to replenish these components of the hair. It will also be appreciated by those skilled in the art that very short application periods are also within the scope of the present invention. That is, a very short application period may be desirable when only a modest modification of the natural hair configuration is to be achieved. Accordingly, all application periods are within the scope of the present invention. Applicants have found, however, that based primarily upon convenience considerations, it is preferred to use the following application periods according to the following approximate hair types:

| Hair Type | Approximate Application Period-Minutes |
|---|---|
| Fine Hair | 10 to 12 |
| Medium Hair | 15 to 20 |
| Coarse Hair | 20 to 30 |

Another step according to the methods of the present invention comprises placing the hair in the desired configuration. Many particular techniques are well known and available in the art for placing the hair in a variety of different configurations. Although the placing step of the present invention may take place before, during or after the application period, it is generally preferred practice when straightening kinky or curly hair to place the hair in the desired configuration only after the application period has expired. That is, the hair will generally not be manipulated during the application period. When the present invention is used in the treatment of hair having a naturally straight configuration, however, it is generally preferred that the hair be placed in the modified configuration either before or during the application of the softening composition of the present invention. It is believed that the details of the procedures used in any particular case to achieve the desired reconfiguration of the hair will be available and well known to those skilled in the art. In applications requiring curling or waving naturally straight hair, for example, it is anticipated that the methods of the present invention will include coiling or wrapping the hair around curlers or rods after the softening agent has been applied.

The methods of the present invention also include testing the effectiveness of the softening process. In certain preferred embodiments, especially those embodiments for the straightening of kinky or curly hair, the testing step of the present invention includes running a fine tooth comb through the hair and observing the resiliency of the hair. If the comb moves through the hair with the desired degree of resistance, this is an indication that the softening process has had the desired degree of effectiveness. Depending upon the particular hair type, the extent of desired straightening, and other factors, this step may last a few seconds to several minutes.

According to a preferred aspect of the present invention, the methods of the present invention further include the step of oxidizing the hair which has received the softening composition and which has been placed in the new configuration. This oxidation step can comprise exposing the hair to air or oxygen. In certain other preferred embodiments, the hair is oxidized by contacting it with a chemical oxidizing agent or neutralizer. The oxidization step of the present invention “quenches” the activity of the softening composition. That is, by exposing the softening solution to oxidation, the capacity of the composition to soften the hair is reduced or eliminated. In this way, the precise amount of softening required can be controlled. The oxidation step also aids in the replacement of the disulfide cystine bonds, which help give the hair its shape. As discussed earlier, application of the softening composition causes cleavage of these disulfide bonds and, on a macroscopic scale, renders the hair relatively malleable. While not intending to be bound by or to any particular theory, applicant believes that such malleability occurs because at least a portion of the individual protein chains, which make up the hair are “decoupled” and allowed to more easily move or slide relative to one another when the disulfide bonds are broken. When the hair is thus subject to the stress caused by placing the hair in the desired configuration, this stress is relieved by the movement of the individual protein chains with respect to one another. The oxidation step according to the methods of the present invention allows such disulfide bonds to be reestablished, thus effecting a permanent reconfiguration of the hair. Standard neutralizing agents are available and well known in the art and the use of all such neutralizing agents are accordingly within the scope of the present invention. Applicant has found, however, that it is preferred to select neutralizing agents from the group consisting of hydrogen peroxides and metal bromate salts, preferably potassium and sodium bromates.

Other well-known hair treatment steps may be preferably used in conjunction with the method steps described above. For example, in certain preferred embodiments, the hair is shampooed prior to application of the present invention. Shampooing in this manner removes fatty acids and oils from the hair and allows enhanced penetration of the softening composition. In a like manner, it may also be preferred to condition the hair prior to the application of the softening composition. In certain embodiments, it is also preferred that the hair be rinsed with tepid water after the application period has expired. In general, the solution, which is rinsed from the hair, will become clear when the rinsing step is complete. More specifically, the rinsing step is expected to last approximately five minutes. In an analogous manner, it is preferred that the hair is also rinsed after the neutralization step is complete. In order to improve the longevity of the curling and straightening process, applicants have also found that it may be desirable to spray the hair after neutralization with an ammonium sulfate solution.

While many techniques are known and available for effectively producing compositions of the present invention, applicant has found that certain preparation methods are preferred for compositions in which the active shaping compound comprises cysteine in major proportion. In particular, it is preferred that cysteine be introduced into solution in the form of hydrated L-cysteine hydrochloride since such material is readily available and contains a relatively precisely known number of milliequivalents of thiol per gram. Due to the acidic nature of hydrated L-cysteine hydrochloride in solution, however, it is preferred that a metal hydroxide be added to the solution during preparation to neutralize the acid component of the L-cysteine hydrochloride. For like reason, it is preferred to add a metal hydroxide to neutralize the acid component when dissolving the cysteamine hydrochloride. Accordingly, it is preferred that potassium or sodium hydroxide be added to the solution in an amount sufficient to provide the required number of milliequivalents of hydroxide to neutralize the acid component of the L-cysteine hydrochloride. In the methods of the present invention for preparing the softening compositions thereof, it is also preferred to introduce a standard anti-oxidant into solution during the preparation process. Although such anti-oxidants are not generally effective for stabilizing the solution, they are beneficial in that they act as scavengers for the oxygen which is introduced into the solution during the preparation process.

### VI. EXAMPLES

The invention will now be further described with reference to the following illustrative but non-limiting examples.

### Example 1A

The composition reported under column A in Table 1 is prepared. In particular, a liquid dye base comprising dye intermediate, carrier and maintaining agent consisting of D-glucose and sucrose in 1:2 weight ratio is prepared in accordance with the composition indicated in Table 2. The composition described in Table 2 consists of about 1.0 parts by weight (PBW) of oxidative dyeing agent, about 97 PBW of carrier and about 2 PBW of maintaining agent. The dye intermediate is described in Table 3.

A bleach booster powder consisting of about 36 PBW of potassium persulfate, about 21 PBW of ammonium persulfate, about 36 PBW silica thickening agent, about 5 PBW of carboxymethylcellulose (CMC-7H3SF), about 1 PBW of fumed silica (CAB-O-SIL M5), and about 1 PBW of chelating agent (Hamp-ene acid) is prepared.

A developer (oxidizing agent) for the dye intermediate GB comprising a 3 percent by weight solution of hydrogen peroxide is provided. The developer thus comprises about 3 PBW of active coloring compound and about 97 PBW of carrier.

A coloring composition according to the present invention is prepared just prior to use by combining about 200 PBW of the dye base with about 30 PBW of the bleach booster powder and about 100 PBW of the developer. The coloring composition thus produced is described under column heading A in Table 1.

The coloring composition is applied to the hair of a human female subject having level two brown color hair by working it into a rich lather on the head of the female subject. After working the composition into the hair for about 3-5 minutes so as to ensure uniform application of the composition evenly to all of the hair of the subject, the composition is allowed to remain on the hair an additional 10 minutes. The hair of the subject is then rinsed thoroughly with water and allowed to dry. Rich, deep tones are produced, a brassy appearance is avoided, and lift, which is about 35% greater, than obtained with other compositions is achieved. The hair is observed to be relatively undamaged by the treatment, and no irritation of the scalp of the subject or the skin of the hair professional is reported. Furthermore, the hair so treated has applied to it two times at day for a month, for a total of 56 applications, a conventional shampoo and conditioner. After such extensive post treatment shampooing and conditioning, the color of the hair remains true and clean, and exhibits a degree of fading of only about 5%.

### Example 1B

The composition reported under column B in Table 1 is prepared. In particular, a liquid dye base comprising dye intermediate, carrier and maintaining agent as in Example 1A is prepared in accordance with the composition indicated in Table 2. The composition described in Table 2 consists of about 1.0 parts by weight (PBW) of oxidative dyeing agent, about 97 PBW of carrier and about 2 PBW of maintaining agent. The dye intermediate is described in Table 3.

A bleach booster powder consisting of about 36 PBW of potassium persulfate, about 21 PBW of ammonium persulfate, about 36 PBW silica thickening agent, about 5 PBW of carboxymethylcellulose (CMC-7H3SF), about 1 PBW of fumed silica (CAB-O-SIL M5), and about 1 PBW of chelating agent (Hamp-ene acid) is prepared.

A developer (oxidizing agent) for the dye intermediate GB comprising a 3 percent by weight solution of hydrogen peroxide is provided. The developer thus comprises about 3 PBW of active coloring compound and about 97 PBW of carrier.

A coloring composition according to the present invention is prepared just prior to use by combining about 100 PBW of the dye base with about 30 PBW of the bleach booster powder and about 200 PBW of the developer. The coloring composition thus produced is described under column heading B in Table 1.

The coloring composition is applied to the hair of a human female subject having level two brown color hair by working it into a rich lather on the head of the female subject. After working the composition into the hair for about 3-5 minutes so as to ensure uniform application of the composition evenly to all of the hair of the subject, the composition is allowed to remain on the hair an additional 10 minutes. The hair of the subject is then rinsed thoroughly with water and allowed to dry. Rich, deep tones are produced, a brassy appearance is avoided, and lift, which is about 35% greater, than obtained with other compositions is achieved. The hair is observed to be relatively undamaged by the treatment, and no irritation of the scalp of the subject or the skin of the hair professional is reported. The richness and depth of color achieved in the hair is maintained at a high level even after repeated shampooing and conditioning subsequent to the treatment.

### Example 1C

The composition reported under column C in Table 1 is prepared. In particular, a liquid dye base comprising dye intermediate, carrier and maintaining agent is prepared in accordance with the composition indicated in Table 2. The composition described in Table 2 consists of about 1.0 parts by weight (PBW) of oxidative dyeing agent, about 97 PBW of carrier and about 2 PBW of glucose. The dye intermediate is described in Table 3.

A bleach booster powder consisting of about 36 PBW of potassium persulfate, about 21 PBW of ammonium persulfate, about 36 PBW silica thickening agent, about 5 PBW of carboxymethylcellulose (CMC-7H3SF), about 1 PBW of fumed silica (CAB-O-SIL M5), and about 1 PBW of chelating agent (Hamp-ene acid) is prepared.

A developer (oxidizing agent) for the dye intermediate GB comprising a 12 percent by weight solution of hydrogen peroxide is provided. The developer thus comprises about 12 PBW of active coloring compound and about 88 PBW of carrier.

A coloring composition according to the present invention is prepared just prior to use by combining about 100 PBW of the dye base with about 30 PBW of the bleach booster powder and about 400 PBW of the developer. The coloring composition thus produced is described under column heading A in Table 1.

The coloring composition is applied to the hair of a human female subject having level two brown color hair by working it into a rich lather on the head of the female subject. After working the composition into the hair for about 3-5 minutes so as to ensure uniform application of the composition evenly to all of the hair of the subject, the composition is allowed to remain on the hair an additional 10 minutes. The hair of the subject is then rinsed thoroughly with water and allowed to dry. Rich, deep tones are produced, a brassy appearance is avoided, and lift, which is about 35% greater, than obtained with other compositions is achieved. The hair is observed to be relatively undamaged by the treatment, and no irritation of the scalp of the subject or the skin of the hair professional is reported. The richness and depth of color achieved in the hair is maintained at a high level even after repeated shampooing and conditioning subsequent to the treatment.

### Example 1D

The composition reported under column C in Table 1 is prepared. In particular, a liquid dye base comprising dye intermediate, carrier and maintaining agent is prepared in accordance with the composition indicated in Table 2, except that the maintaining agent is present in an amount of 5 percent by weight and the amount of water is about 3 percent by weight less. Thus, the composition described in Table 2 is modified to consist of about 1.0 parts by weight (PBW) of oxidative dyeing agent, about 94 PBW of carrier and about 5 PBW of maintaining agent. The dye intermediate is described in Table 3.

A bleach booster powder consisting of about 36 PBW of potassium persulfate, about 21 PBW of ammonium persulfate, about 36 PBW silica thickening agent, about 5 PBW of carboxymethylcellulose (CMC-7H3SF), about 1 PBW of fumed silica (CAB-O-SIL M5), and about 1 PBW of chelating agent (Hamp-ene acid) is prepared.

A developer (oxidizing agent) for the dye intermediate GB comprising a 3 percent by weight solution of hydrogen peroxide is provided. The developer thus comprises about 3 PBW of active coloring compound and about 97 PBW of carrier.

A coloring composition according to the present invention is prepared just prior to use by combining about 200 PBW of the dye base with about 30 PBW of the bleach booster powder and about 100 PBW of the developer. The coloring composition thus produced is described under column heading D in Table 1 The coloring composition is applied to the hair of a human female subject having level two brown color hair by working it into a rich lather on the head of the female subject. After working the composition into the hair for about 3-5 minutes so as to ensure uniform application of the composition evenly to all of the hair of the subject, the composition is allowed to remain on the hair an additional 10 minutes. The hair of the subject is then rinsed thoroughly with water and allowed to dry. Rich, deep tones are produced, a brassy appearance is avoided, and lift, which is about 35% greater, than obtained with other compositions is achieved. The hair is observed to be relatively undamaged by the treatment, and no irritation of the scalp of the subject or the skin of the hair professional is reported. The richness and depth of color achieved in the hair is maintained at a high level even after repeated shampooing and conditioning subsequent to the treatment.

TABLE 1
LIGHT BROWN COLORING COMPOSITION

| | Concentration, w % | | | |
|---|---|---|---|---|
| Component | A | B | C | D |
| Oxidizing Agent | | | | |
| K₂S₂O₈ | 3.3 | 3.3 | 2.3 | 3.3 |
| (NH₄)₂S₂O₈ | 1.9 | 1.9 | 1.2 | 1.9 |
| H₂O₂ | 0.9 | 1.8 | 2.4 | 1.8 |
| Carrier | | | | |
| H₂O | 48 | 68 | 76 | 45.8 |
| Isopropyl Alcohol and | 11.9 | 5.9 | 4.0 | 11.8 |
| Hexelene Glycol | | | | |
| Alkalizer | 1.5 | 0.8 | 0.5 | 1.5 |
| Detergent | 4.6 | 2.3 | 1.5 | 4.6 |
| Spreading Agent | 7.8 | 3.9 | 2.6 | 7.7 |
| Anti-oxidant | 0.5 | 0.2 | 0.2 | 0.5 |
| Chelating Agent | 0.2 | 0.2 | 0.1 | 0.2 |
| Thickener | 10.8 | 7.3 | 4.9 | 10.7 |
| Penetrating Agent | 6.8 | 3.4 | 2.3 | 6.7 |
| Dye Intermediate GB | 0.6 | 0.3 | 0.2 | 0.6 |
| Glucose | 1.2 | 0.6 | 0.4 | 3.0 |
| | 100.0 | 100 | 100 | 100 |
| Ratio | | | | |
| Maintaining agent:Active | 0.18:1 | 0.08:1 | 0.08:1 | 0.39:1 |
| Coloring Compound | | | | |

TABLE 2
DYE BASES

| Component | Concentration, Wt % |
|---|---|
| Dye Intermediate | 1.0 |
| Carrier | |
| Deionized Water | 30.55 |
| Isopropyl Alcohol | 9.8 |
| Urea | 2.5 |
| Oleic Acid | 7.6 |
| Hexelene Glycol | 9.8 |
| Lauryl Alcohol | 11.5 |
| Octylphenoxypoly (ethyleneoxy) | 12.8 |
| ethanol | |
| NaSO₄ | 0.75 |
| Monoethanol Amine | 11.2 |
| EDTA | 0.25 |
| Glucose | 2 |
| | 100.0 |

TABLE 3
DYE INTERMEDIATE

| Component | Concentrations, PBW |
|---|---|
| p-phenylenediamine | 29.7 |
| N,N,-diethyl-n-amino SO₄ | 5.8 |
| p-nitro-o-phenylenediamine | |
| m-Aminophenol | 0.4 |
| o-Aminophenol | 0.3 |
| 2-methylresorcinol | 14.1 |
| Resorcinol | 29.8 |
| 5-nitro-2-aminophenol | |
| p-Aminophenol and p-o-nitro-p- | 0.4 |
| phenylenediamine | |
| 2-amino-5-nitro phenol | 0.7 |
| N,N Bis(2-hydroxyethyl)-p- | 2.1 |
| phenylenediamine sulfate | |
| 4-ethoxy-m-phylenediamine SO₄ | 4.1 |

### Example 2

### Normal Perm Composition

To a clean, disinfected tank equipped with a mixer, are added 5.5401 pounds of deionized water and then, while mixing, sufficient amounts of maltose to achieve about 7% by weight of maltose in the final composition. Stirring is continued until all the maltose is dissolved. Then, while mixing, 0.91 pounds of Dequest 2016 (a sodium aminotrimethylene phosphonate chelating agent) is added, followed by 0.7826 pounds of 28% ammonium hydroxide and 0.0910 pounds of Thioset M (a 50% solution of monoethanolamine sulfate) and then 0.6916 pounds of anhydrous L-cysteine hydrochloride. Stirring is continued until complete solution is achieved; then 1.0374 pounds of cysteamine hydrochloride and 0.0018 pounds of sodium bisulfite are added and stirring continued until complete solution is achieved. In a separate stainless steel container, 0.364 pounds of Lipicol L-23 surfactant (Laureth 23) is heated to 50° C. Then the fragrance is added while mixing is continued until a clear pre-mix solution of fragrance is obtained. Then the pre-mix solution of fragrance is added to the tank and stirring continued for 30 minutes after complete solution is achieved. The foregoing ingredients and their weight percentage of the total composition are listed in Table I below.

The resulting solution is clear, has a pH in the range of 8.35-8.55 at a temperature of about 25° C., an alkali content of about 0.90 to 1.10% by weight and a thio (SH) content of about 1.5 to about 1.7% by weight.

TABLE I

| Description | Quantity (LB) | % By Weight |
|---|---|---|
| Phosphonic acid (1-hydroxyethylidene) | | 2. |
| bis-, tetrasodium salt | | |
| Aqueous Ammonia (28%) | | 8.6 |
| L-Cysteine HCl Anhydrous | | 7.6 |
| Cysteamine HCl | | 11.4 |
| Maltose | | 7. |
| Laureth-23 | | 4. |
| Dequest 2016 | | 1. |
| Fragrance | | 0.5 |
| Deionized Water | | balance |

### Example 3

### Bleach Oil

A bleach oil composition may be made with the following composition.

TABLE II

| Description | % By Weight |
|---|---|
| Sodium Sulfite | 0.5 |
| Trisodium Phosphate | 1.5 |
| Maltose, Glucose or a combination | 5. |
| Isopropyl Alcohol | 9.6 |
| Ethanolamine | 5.3 |
| Oleic Acid | 7.1 |
| Octoxynol-9 | 8.3 |
| Lauramidopropyl Betaine | 9.3 |
| Cocamide DEA | 5.4 |
| Lauryl Alcohol | 11.4 |
| Propylene Glycol | 4.8 |
| Disodium EDTA | 0.25 |
| Sorbotol | 9.2 |
| Deionized Water | balance |

### Example 4

### Bleach Oil

A relaxer composition may be made with the following composition.

TABLE III

| Description | % By Weight |
|---|---|
| Sodium Hydroxide | 2.5 |
| Maltose, Glucose and/or combination | 5. |
| Cetearyl Alcohol and Cetearyl Phosphate | 7. |
| Mineral Oil | 8. |
| Behentrimonium Methoslfate and | 1. |
| Ceteryl Alcohol | |
| Stearyl Alcohol | 1. |
| Stereth - 10 | 2.5 |
| Sterth - 2 | 0.5 |
| DEA Oleth-10 Phosphate | 0.75 |
| Propylene Glycol | 3. |
| Fragrance | 0.2 |
| Deionized Water | balance |

It will be evident to those skilled in the art that the invention is not limited to the details of the foregoing illustrative examples and that the present invention may be embodied in other specific forms without departing from the essential attributes thereof, and it is therefore desired that the present embodiments and examples be considered in all respects as illustrative and that it will be understood that variations can be made without departing from the spirit and scope of the invention.

1. A composition for treating hair or skin comprising:
a) at least one active treatment compound;
a) a carrier for said active treatment compound; and
c) a maintaining agent comprising a reducing saccharide and non-reducing saccharide in a reducing saccharide:non-reducing saccharide weight ratio of from about 9:1 to about 1:2.

2. The composition of claim 1 wherein said at least one treatment compound comprises an alkalizing agent.

3. The composition of claim 1 wherein said alkalizing agent comprises ammonia.

4. The composition of claim 2 wherein said non-reducing saccharide comprises sucrose.

5. The composition of claim 1 wherein said reducing saccharide is selected from the group consisting of maltose, lactose, glucose, fructose or combinations of two or more of these.

6. A shampoo comprising the composition of claim 1.

7. A conditioner comprising the composition of claim 1.

8. A permanent wave composition comprising the composition of claim 1.

9. The composition of claim 1 wherein said maintaining agent is present in the composition in an amount of from about 0.5% to about 15% by weight.

10. The composition of claim 1 wherein said maintaining agent is present in the composition in an amount of from about 1% to about 5% by weight.

11. The composition of claim 1 wherein said reducing saccharide:non-reducing saccharide weight ratio is from about 5:1 to about 1:2.

12. A method of treating hair to produce a lasting effect on at least one property of the hair comprising:
a) applying to the hair at least one active compound having an effect on at least one physical property of the hair; and
b) before, during or after said applying step a), applying a maintaining agent to the hair, said maintaining agent comprising a reducing saccharide and non-reducing saccharide in a reducing saccharide:non-reducing saccharide weight ratio of from about 9:1 to about 1:2.

13. The method of claim 12 wherein said physical property comprises the color of the hair.

14. The method of claim 12 wherein said physical property comprises the shape of the hair.

15. The method of claim 12 wherein said physical property comprises the cleanliness of the hair.

16. The method of claim 12 wherein the physical property of the hair is maintained for a period of time, which is at least about 30% greater than the amount of time, said property would be maintained in the absence of said maintaining agent.

17. The method of claim 12 wherein said maintaining step b) comprises combining said maintaining agent with said active compound.

18. The method of claim 12 wherein said active compound is included in a coloring agent.

19. The method of claim 12 wherein said active compound is included in a hair shaping composition.

20. The method of claim 19 wherein said hair shaping composition comprises a shaping agent.

21. The method of claim 20 wherein said shaping agent comprises cysteamine from about 10 to about 25% by weight of active shaping compound.

22. The method of claim 12 wherein the reducing saccharide:non-reducing saccharide weight ratio is from about 5:1 to about 1:2
